(19) United States
(12) Patent Application Publication
(10) Pub. No.: US 2008/0255573 A1
(43) Pub. Date: Oct. 16, 2008

(54) SURGICAL LASER TRAJECTORY INSTRUMENT

(75) Inventors: Keith Willett, Oxford (GB); Rebecca Parrott, Warsaw, IN (US); Stephanie Boettcher, Houghton, MI (US); Kerry Bodle, South Bend, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/054,710

(22) Filed: Mar. 25, 2008

Related U.S. Application Data
(60) Provisional application No. 60/922,767, filed on Apr. 10, 2007.

(51) Int. Cl. A61B 17/56

(52) U.S. Cl. 606/96

(57) ABSTRACT

A system for illuminating patient anatomy to locate a site for an incision.

### PRIORITY

The present application claims the priority of U.S. Provisional Application Ser. No. 60/922,767 filed Apr. 10, 2007, the disclosure of which is incorporated herein by reference.

### BACKGROUND AND SUMMARY

The present invention relates to systems for fixing bone, and, more particularly, to an instrument for identifying the location on the tissue for placement of bone fasteners.

The present disclosure provides, a guidance system comprising a light-emitting device configured to be attached to a guide that is coupled to a bone fastener.

Another embodiment of the present disclosure provides a guidance system comprising a housing, a power source, and means for identifying a location on anatomy of a patient between a guide and a nail.

Yet another embodiment of the present disclosure provides a method of fixing a bone comprising the steps of placing a nail within the bone; placing a light source in contact with a guide; and using the light source to identify an incision site for fixing the nail.

### BRIEF DESCRIPTION OF THE DRAWINGS

The above-mentioned and other features of the disclosure, and the manner of attaining them, will become more apparent and better understood by reference to the following description of an embodiment of the disclosure taken in conjunction with the accompanying drawings, wherein:

FIG. 1 is a perspective and partially transparent view of a laser in use according to the present disclosure;

FIG. 2 is a perspective and partially transparent view of the anatomy of FIG. 1 with a cannula inserted;

FIG. 3 is a cross-sectional view of a laser disposed in a housing;

FIG. 4 is a perspective view of the laser of FIG. 1;

FIG. 5 is a cut-away view of another embodiment of a laser disposed in a housing according to the present disclosure; and

FIG. 6 is a cut-away view of yet another embodiment of a laser disposed in a housing according to the present disclosure.

Corresponding reference characters indicate corresponding parts throughout the several views. The exemplification set out herein illustrates one preferred embodiment of the invention, in one form, and such exemplification is not to be construed as limiting the scope of the invention in any manner.

### DETAILED DESCRIPTION OF THE INVENTION

The description that follows refers to a retrograde femoral nail application. While described with respect to a retrograde femoral nail application, the principles of the present disclosure can be applied to other surgical applications.

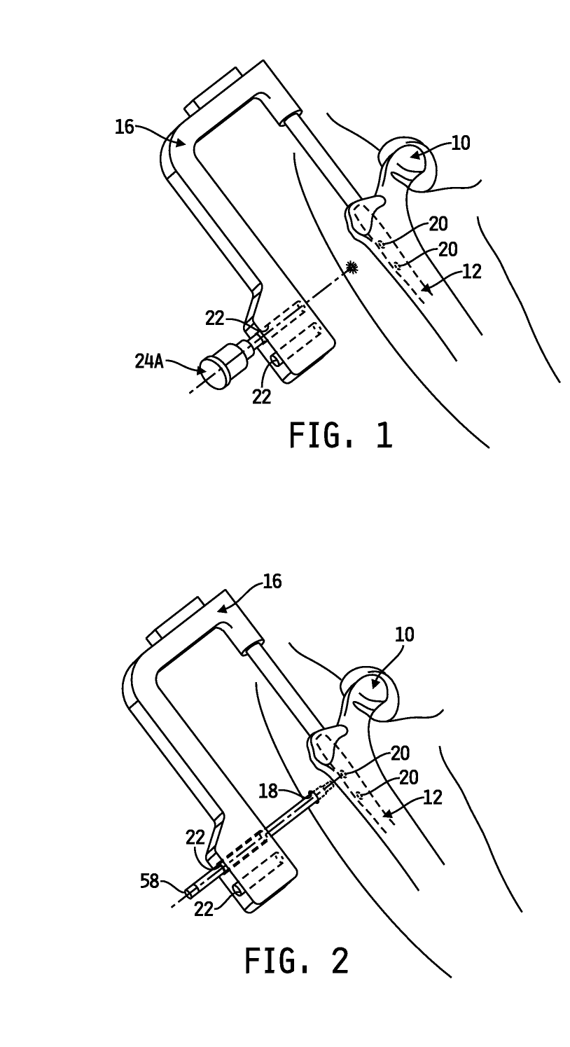

FIG. 1 shows femur 10 having intermedullary nail 12 inserted therein. One of laser devices 24a-c is provided to indicate a location on the skin of a patient for a user to make an incision for the insertion of a screw (not shown) to secure or fix nail 12. Nail 12 is available in a plurality of diameters such that one may be selected that is appropriate for the anatomy of the intended patient.

Placement of nail 12 in a patient presenting a femoral fracture includes several steps, not all of which will be discussed herein, but are known to those of ordinary skill in the art. Before beginning the surgical procedure, the fracture is reduced.

An incision is made just posterior to midline of trochanter and just medial to prominence of trochanter in trochanteric fossa. Next, a proper entry portal into intramedullary canal 102 is located utilizing a pin (not shown) and a reamer (not shown).

Reaming is then performed along the canal in a way customized appropriately to the anatomy of the patient. The reamer is then removed.

Nail 12 is then placed over the guide wire and inserted into femur 10. Once so inserted, targeting guide 16 is attached to nail 12 and utilized to drill transverse bores 18 into femur 10 and soft tissue that align with bores 20 within nail 12. Alternatively, guide 16 is attached to nail 12 prior to insertion of nail 12 into the anatomy. Before bores 18 are drilled, incisions are made through the skin of the patient. Targeting guide 16 is also used to insert a plurality of condyle screws (not shown) through transverse bores 20 via a cannula 58. Any suitable targeting guide may be used, such as the guide disclosed in U.S. Pat. No. 5,178,621, assigned to Zimmer, Inc., the disclosure of which is incorporated herein by reference.

Targeting guide 16 includes bores 22 proximate the distal end thereof. Bores 22 are located so as to align with bores 20 of nail 12 to which guide 16 is attached. One of laser devices 24a-c is provided to show the user where on the skin of a patient an incision should be made to allow placement of the screws to secure nail 12. Laser devices 24a-c fit within either of bores 22. When activated, laser devices 24a-c illuminate the skin to indicate the proper incision site. The surgeon/user then makes an incision at the indicated site. Femoral screw bushing or other cannula 58 is then inserted into bores 22 to provide a guide for other bushings and/or bits to drill a hole aligned with bores 20 of nail 12. Ultimately, screws (not shown) are affixed through bores 20 to fix nail 12 to femur 10.

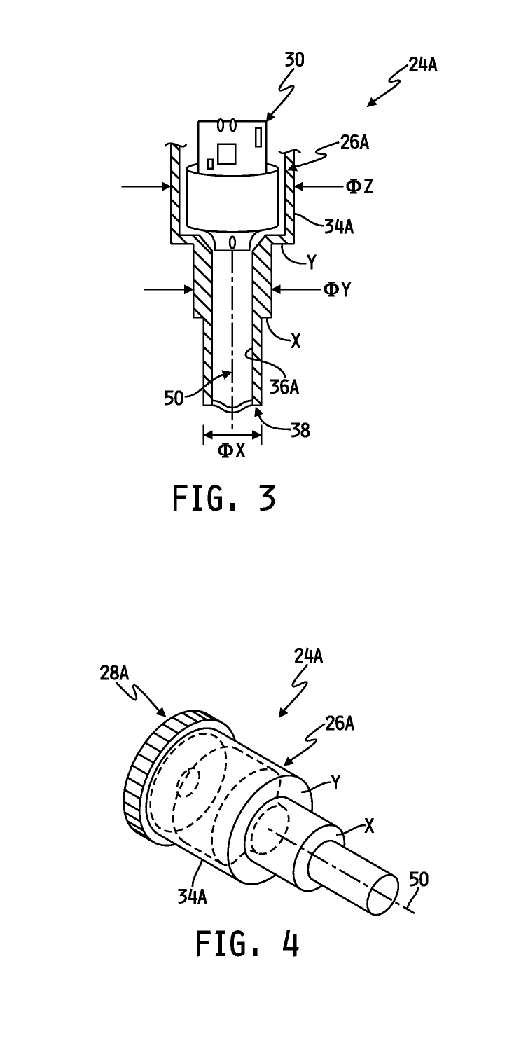

Three embodiment laser devices 24a-c are shown in FIGS. 3-6. Each laser device 24a-c includes outer housing 26a-c, casing cap 28a-c, laser module 30, and battery pack 32.

Device 24a, as shown in FIG. 3, includes modular outer housing 26a. Housing 26a includes multi-diametered outer surface 34a having diameters indicated as ØX, ØY, and ØZ. multi-diametered outer surface 34a is sized and shaped to fit in various sized bores of various targeting guides 16. For example, diameters ØX, ØY, and ØZ may be 6, 8, and 11 mm respectively to match up with multiple similarly sized screw cannulas. For example, when engaging 6 mm screw cannulas (not shown), device 24a may enter the 6 mm cannula until shoulder X is abutted by the cannula. Similarly, 8 mm cannulas receive device 24a until shoulder Y is abutted and 11 mm cannulas receive device 24a until cap 28a is abutted. Housing 26a includes inner bore 36a sized to receive laser module 30 and battery pack 32 (not shown in FIG. 3) therein. Inner bore 36a also includes a threaded portion (not shown) that engages similar threads on casing cap 28a. It should be appreciated that housing 26a may alternatively attach to casing cap 28a via threading on the exterior of housing 26a, mating lips, or any other suitable means. Inner bore 36a further includes distal end 38 that allows the projection of laser beam 50 therefrom. Laser beam 50 is centered relative to housing 26a and follows longitudinal axis 51 of housing 26a. Laser device 24a is toggled between an on and off setting via a button (not shown) located on cap 28a. The button is similar to the button found on a retractable ball-point pen in that pushing the button once allows power to be sent to laser module 30 and pushing the button a second time disengages power from laser module 30. Alternatively, any other suitable means of selectively providing power to laser module 30, such as those described below, may be used.

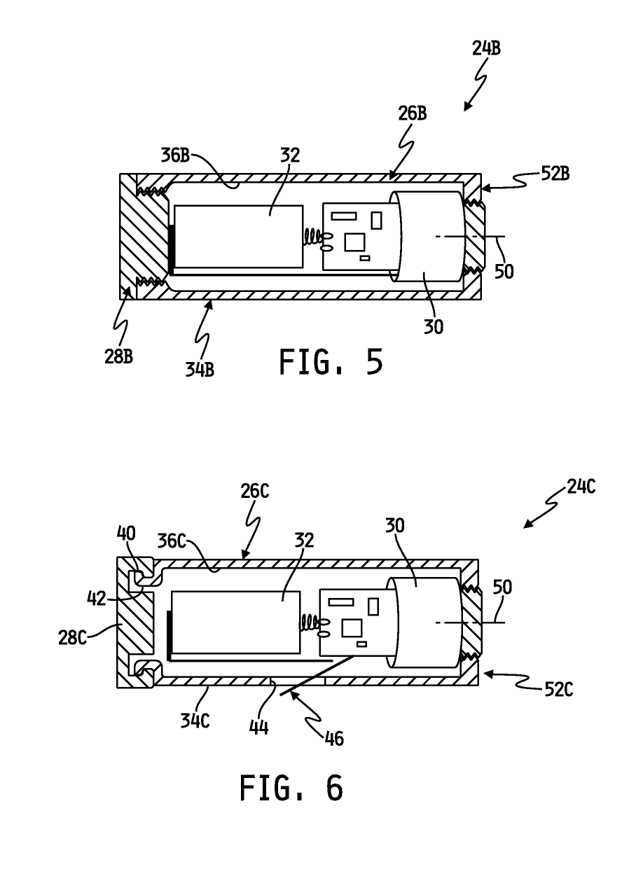

Device 24b, as shown in FIG. 5, includes substantially cylindrical housing 26b. Housing 26b includes outer surface 34b sized and shaped to fit in bores 22 of targeting guide 16. Housing 26b includes inner bore 36b sized to receive laser module 30 and battery pack 32 therein. Laser module 30 is positioned to emit laser beam 50 from distal end 52b of housing 26b. Inner bore 36b also includes a threaded portion 37 that engages similar threads 39 on casing cap 28b. Fully tightening casing cap 28b into housing 26b completes an electrical circuit between laser module 30 and battery pack 32, thereby turning on device 24b.

Device 24c, as shown in FIG. 6, includes substantially cylindrical housing 26c. Housing 26c includes outer surface 34c sized and shaped to fit in bores 20 of targeting guide 16. Housing 26c includes inner bore 36c sized to receive laser module 30 and battery pack 32 therein. Laser module 30 is positioned to emit laser beam 50 from distal end 52c of housing 26c. Outer surface 34c also includes shoulders or lips 40 that engages similar shoulders or lips 42 on casing cap 28c. Outer surface 34c also includes void 44 that provides for switch 46 to be positioned therein. Switch 46, in an off position, extends slightly outside of the outer surface 34c. Pressing switch 46 such that switch 46 is even with outer surface 34c allows the completion of an electrical circuit between battery pack 32 and laser module 30. Accordingly, placing device 24c within either of bores 22 causes switch 46 to be depressed and causes activation of laser device 24c. Thus, laser device 24c is activated for the time that device 24c remains inserted in any one of bores 22.

FIG. 2 shows inserted nail 12 with guide 16 attached thereto. In use, devices 24a-c are inserted into one of bores 22. Once placed, device 24a-c is either manually or automatically activated to emit laser beam 50. Laser beam 50 thus projects spot 56 on the skin of the patient at the appropriate place for an incision. The user then makes such an incision. Device 24a-c is then removed and either manually or automatically deactivated and the surgeon/user proceeds with the surgery.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A guidance system comprising:
a light-emitting device configured to be attached to a guide that is coupled to a bone fastener.

2. The guidance system of claim 1, wherein the light-emitting device is sized and shaped to be received in a bore defined in the guide.

3. The guidance system of claim 1, wherein the light-emitting device is configured to illuminate a part of the anatomy intermediate the bone fastener and the guide when the bone fastener is in anatomy of a patient.

4. The guidance system of claim 1, wherein the light-emitting device is configured to be activated by attaching the light-emitting device to the guide.

5. The guidance system of claim 1, wherein the light-emitting device includes a housing having sections of differing external profiles configured to be received in bores having differing internal profiles defined in guides.

6. The guidance system of claim 1, wherein the light-emitting device is configured to be activated by tightening a cap of a housing of the light-emitting device.

7. The guidance system of claim 1, wherein the light-emitting device is configured to emit a laser beam along a longitudinal axis of the light-emitting device.

8. A guidance system comprising:
a housing;
a power source; and
means for identifying a location on anatomy of a patient between a guide and a nail.

9. The guidance system of claim 8, wherein the location is between a bore in the guide and a bore in the nail.

10. The guidance system of claim 8, wherein the means for identifying includes a laser.

11. The guidance system of claim 8, wherein the means for identifying includes a housing configured to couple to the guide.

12. The guidance system of claim 8, wherein the identifying means includes a light source and activating means for activating the light source.

13. The guidance system of claim 12, wherein the activating means includes a switch configured to engage the guide.

14. The guidance system of claim 12, wherein the activating means includes a switch disposed on a cap of the identifying means.

15. A method of fixing a bone including the steps of:
placing a nail within the bone;
placing a light source in contact with a guide; and
using the light source to identify an incision site.

16. The method of claim 15, wherein the nail includes a bore and the step of placing a light source in contact with the guide causes the light source to point to the bore.

17. The method of claim 15, wherein the guide includes a bore configured to receive the light source therein.

18. The method of claim 17, wherein the step of placing a light source in contact with the guide includes placing the light source within the bore.

19. The method of claim 18, wherein the step of placing the light source in contact with the guide causes the light source to illuminate a location on a patient's anatomy.

20. The method of claim 15, wherein the step of placing a light source in contact with the guide includes automatically activating the light source.
